(19) United States
(12) Patent Application Publication
(10) Pub. No.: US 2022/0197021 A1
(43) Pub. Date: Jun. 23, 2022

(54) METHOD OF MANUFACTURING ACHROMATIC LENS

(71) Applicant: MPNICS CO., LTD., Gwangju (KR)

(72) Inventors: Sang Do KANG, Gwangju (KR); Seung Keun OH, Gwangju (KR); Hun KIM, Gwangju (KR)

(21) Appl. No.: 17/376,485

(22) Filed: Jul. 15, 2021

(30) Foreign Application Priority Data
Dec. 17, 2020 (KR) 10-2020-0176971

(51) Int. Cl. G02B 27/00; G02B 3/04; B29D 11/00

(52) U.S. Cl. CPC G02B 27/0025; B29D 11/00403; G02B 3/04

(57) ABSTRACT

In a method of manufacturing an achromatic lens according to an embodiment of the present disclosure, the achromatic lens in which a first lens and a second lens are integrally molded is manufactured by using mold processing, so that chromatic aberration correction ability may be improved without using an adhesive or a separate instrument to fix the first lens and the second lens to each other.

### CROSS REFERENCE TO RELATED APPLICATION

The present application claims priority to Korean Patent Application No. 10-2020-0176971, filed Dec. 17, 2020, the entire contents of which is incorporated herein for all purposes by this reference.

### BACKGROUND OF THE INVENTION

### 1. Field of the Invention

The present invention relates to a method of manufacturing an achromatic lens and, more particularly, to a method of manufacturing an achromatic lens, wherein the achromatic lens in which a first lens and a second lens are integrally molded is manufactured by using mold processing, so that chromatic aberration correction ability may be improved without using an adhesive or a separate instrument to fix the first lens and the second lens to each other.

### 2. Description of the Related Art

In general, an achromatic lens is manufactured so that lenses made of glass having different components are combined with each other to cancel each other's chromatic aberration.

Such an achromatic lens is necessary to satisfy complex conditions such as a refractive index of a material, a curvature radius of a lens, and mutual arrangement of each lens, and at the same time, correction of spherical aberration is also required, thereby having many design difficulties.

In addition, since chromatic aberration may not be completely eliminated for all colored lights, the chromatic aberration is usually corrected for representative colored lights selected according to each application. Among these types of lenses, achromat is a lens in which colors are erased for two-color light, and apochromat is a lens in which chromatic aberration is corrected for three-color light. For example, in a photo lens, it is considered such that color erasure is performed on two colors of yellow and indigo light or three colors of light in which reddish light is added to the two colors, whereby the color that visually feels the strongest and the color that is most strongly sensitized to a film are allowed to form an image at the same point.

In order to manufacture an achromatic lens, as shown in FIG. 1, a first lens 11 and a second lens 12 are manufactured, and then the first lens 11 and the second lens 12 are bonded with an epoxy resin 13, so as to be used. Alternately, as shown in FIG. 2, a first lens 11 and a second lens 12 are manufactured, and then by using a separate instrument 14 configured as an open shape at each of opposite ends thereof, the first lens 11 and the second lens 12 are assembled together, so as to be physically tightened, thereby being utilized.

However, in the conventional method of manufacturing an achromatic lens, when manufacturing the achromatic lens by using an epoxy resin, since the epoxy resin 13 is coated between the first lens 11 and the second lens 12, there is a problem in that chromatic aberration occurs depending on physical properties, discharge amount, a bonding error of the epoxy resin 13, and the like. Furthermore, when the achromatic lens is applied to a medical endoscope, there is a problem of raising concerns in which the medical endoscope may be harmful to the human body due to the use of the epoxy resin 13.

In addition, when fixing the first lens 11 and the second lens 12 to each other by using the separate instrument 14, there are problems in that cracks, breaks, and the like may occur because the first lens 11 and the second lens 12 collide with each other, a fine gap may be generated between the first lens 11 and the second lens 12 due to an alignment error, thereby resulting in chromatic aberration, and production cost increases as the separate instrument is used.

### SUMMARY

The present invention has been devised to solve the above-described problems, and an objective of the present invention is to provide a method of manufacturing an achromatic lens, wherein without using a separate instrument or a bonding process that uses an epoxy resin, a first lens and a second lens are integrally molded through mold processing, so that chromatic aberration correction ability may be improved.

The objective of the present invention is not limited to the objectives mentioned above, and other objectives not mentioned herein will be clearly understood from the following description.

In order to achieve the above objective, the present invention provides a method of manufacturing an achromatic lens, the method including: preparing molds comprising a sleeve mold provided with a cylindrical through-hole at a central part thereof, a lower mold fitted at an upper end thereof to a lower part of the through-hole of the sleeve mold, and a first upper mold and a second upper mold pressed downward from an upper part of the sleeve mold; molding a first lens by placing a first lens material on an upper surface of the lower mold and pressing the first upper mold downward; and molding a second lens by placing a second lens material on an upper surface of the first lens and pressing the second upper mold downward, thereby molding the achromatic lens in which the second lens is integrally molded on the first lens.

In a preferred exemplary embodiment, the upper surface of the lower mold may be provided with a planar shape, a convex part having an aspherical-surface shape, or a groove part having an aspherical-surface shape.

In the preferred exemplary embodiment, the lower surface of the first upper mold may be provided with a convex part having an aspherical-surface shape.

In the preferred exemplary embodiment, the lower surface of the second upper mold may be provided with a planar shape, a convex part having an aspherical-surface shape, or a groove part having an aspherical-surface shape.

In the preferred exemplary embodiment, the first lens material and the second lens material may have a difference in coefficient of thermal expansion of less than 1.0×10⁻⁶/° C.

In the preferred exemplary embodiment, a material having a Tg (glass transition temperature) point higher than a Tg point of the second lens material by more than 50 degrees may be used as the first lens material.

In the preferred exemplary embodiment, a difference between a refractive index (Nd) of the first lens material and a refractive index (Nd) of the second lens material may exceed 0.15.

In addition, the present invention further provides an achromatic lens, including: a first lens and a second lens provided integrally therein, wherein the achromatic lens is manufactured by a method of manufacturing the achromatic lens.

In addition, the present invention provides a medical endoscope including an achromatic lens.

The present invention has the following excellent effects.

According to the method of manufacturing an achromatic lens of the present invention, since the first lens and the second lens are integrally molded by mold processing, compared with the conventional method of manufacturing an achromatic lens by using epoxy bonding, a phenomenon in which chromatic aberration occurs depending on physical properties, discharge amount, a bonding error of the epoxy resin, and the like may be eliminated, and compared with the manufacturing method of an achromatic lens by using the conventional instrument, the phenomena in which cracks, breaks, and the like occur due to the first lens and the second lens colliding with each other by the separate instrument, and chromatic aberration is generated due to the alignment error are prevented, whereby there is an effect of having excellent chromatic aberration correction ability.

In addition, according to the method of manufacturing an achromatic lens of the present invention, since the first lens and the second lens are not combined by using an adhesive such as an epoxy resin, when applied to a medical endoscope, there is an effect in that the concerns of harmfulness to the human body may be resolved.

### BRIEF DESCRIPTION OF THE DRAWINGS

FIG. 1 is a view showing a method of manufacturing a conventional achromatic lens using epoxy bonding.

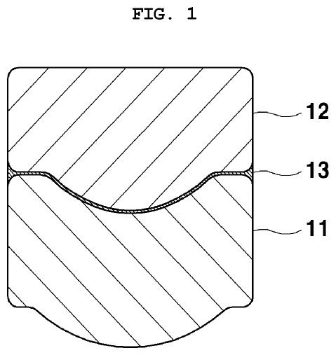

FIG. 2 is a view showing the method of manufacturing the conventional achromatic lens using an instrument.

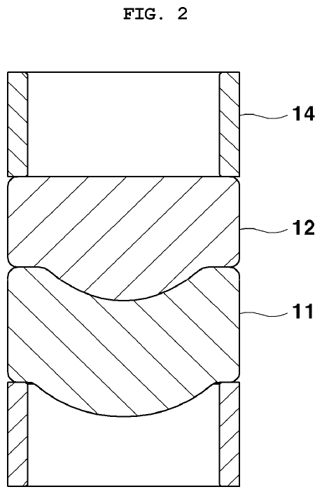

FIG. 3 is a step diagram showing a method of manufacturing an achromatic lens according to the present invention.

FIG. 4 is a schematic diagram showing the method of manufacturing an achromatic lens according to the present invention.

FIG. 5 is a view showing a mold used in the method of manufacturing an achromatic lens according to the present invention.

FIG. 6 is a view showing a photographed image of the achromatic lens manufactured by the method of manufacturing an achromatic lens according to the present invention.

### DETAILED DESCRIPTION

The terms used in the present invention have been selected from general terms that are currently, widely used as much as possible, but in certain cases, there also exists terms that are arbitrarily selected by the applicant. In this case, the meaning should be interpreted by taking into considering the meanings of the terms described or used in the detailed description of the present invention, rather than just by using the names of terms.

Hereinafter, the technical configuration of the present invention will be described in detail with reference to preferred exemplary embodiments illustrated in the accompanying drawings.

However, the present invention is not limited to the exemplary embodiments described herein and may be embodied in other forms. The same reference numerals throughout the specification indicate the same components.

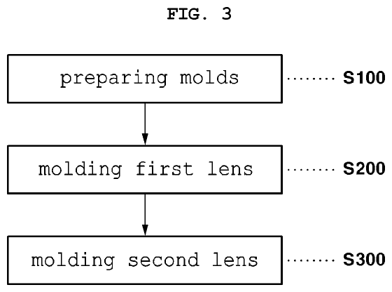

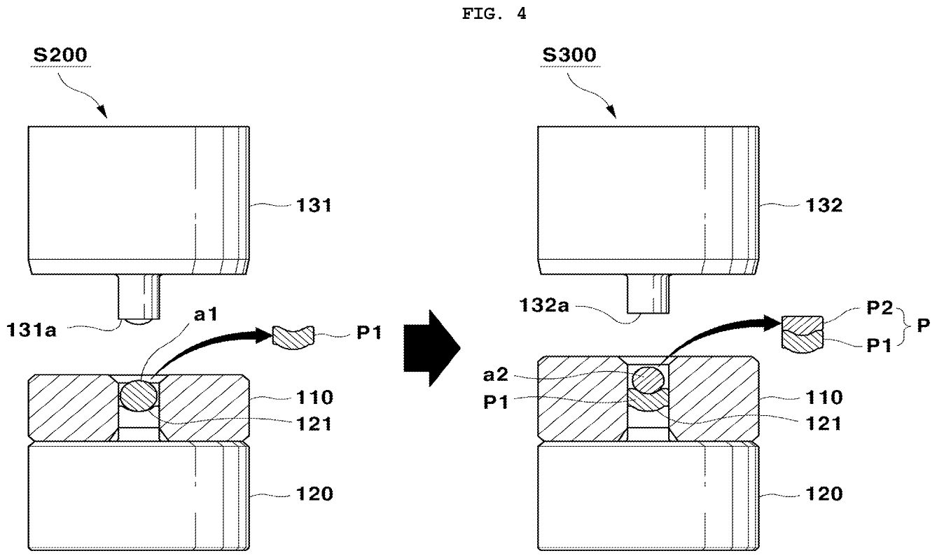

FIG. 3 is a step diagram showing a method of manufacturing an achromatic lens according to the present invention, and FIG. 4 is a schematic diagram showing the method of manufacturing an achromatic lens according to the present invention.

Referring to FIGS. 3 and 4, the method of manufacturing an achromatic lens according to the exemplary embodiment of the present invention is to manufacture the achromatic lens, wherein without using an adhesive or a separate instrument to fix lenses, a first lens and a second lens are integrally molded by using mold processing, thereby having excellent chromatic aberration correction ability. First, step S100 of preparing molds is performed.

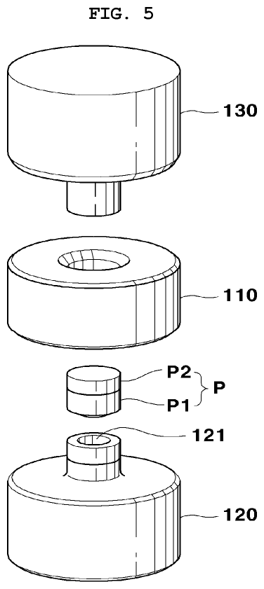

Meanwhile, FIG. 5 is a view showing a mold used in the method of manufacturing an achromatic lens according to the present invention. The mold includes: a sleeve mold 110, a lower mold 120, a first upper mold 131, and a second upper mold 132.

Here, the sleeve mold 110 is provided with a cylindrical through-hole at a central part thereof. That is, the sleeve mold 110 is configured as an open shape at each of opposite ends thereof.

In addition, the lower mold 120 is provided with an upper end thereof fitted to a lower part of the through-hole of the sleeve mold 110.

In addition, an upper surface 121 of the lower mold 120 is provided with a planar shape, or may be formed with a convex part having an aspherical-surface shape or a concave part (i.e., groove part) having an aspherical-surface shape.

In addition, the first upper mold 131 is pressed downward from an upper part of the sleeve mold 110.

Here, a convex part having an aspherical-surface shape is formed on a lower surface 131a of the first upper mold 131.

In addition, similar to the first upper mold 131, the second upper mold 132 is pressed downward from the upper part of the sleeve mold 110.

Here, a lower surface of the second upper mold 132 is provided with a planar shape, or may be formed with a convex part having an aspherical-surface shape or a groove part having an aspherical-surface shape.

Next, step S200 of molding a first lens p1 is performed, wherein a first lens material al is placed on the upper surface 121 of the lower mold 120, and the first upper mold 131 is pressed downward, so as to mold the first lens p1.

Through step S200 of molding a first lens p1, a shape corresponding to a lower surface of the first upper mold 131 is transferred to an upper surface of the first lens p1, and a shape corresponding to the upper surface 121 of the lower mold 120 is transferred to the lower surface of the first lens p1.

That is, a groove part having an aspherical-surface shape is formed on the upper surface of the first lens p1, and a planar shape, a groove part having an aspherical-surface shape, or a convex part having an aspherical-surface shape is formed on the lower surface of the second lens p2.

Next, step S300 of molding a second lens is performed, wherein a second lens material a2 is placed on the upper surface of the first lens p1 molded through step S200 of molding a first lens, the second upper mold 132 is pressed downward, and an achromatic lens p in which the second lens p2 is integrally formed on the first lens p1 is molded. Through step S300 of molding a second lens, the second lens p2 is integrally molded on the upper part of the first lens p1, a shape corresponding to the lower surface of the second upper mold 132 is transferred to the upper surface of the second lens p2, and a shape corresponding to the upper surface of the first lens p1 is transferred to the lower surface of the second lens p2.

That is, a planar shape, a groove part having an aspherical-surface shape, or a convex part having an aspherical-surface shape is formed on the upper surface of the second lens p2, and a convex part having an aspherical-surface shape is formed on the lower surface of the second lens p2.

In addition, it is preferable that a difference in coefficient of thermal expansion between the first lens material al and the second lens material a2 is less than 1.0×10⁻⁶/° C.

The reason is that when the difference in the coefficient of thermal expansion between the first lens material al and the second lens material a2 exceeds 1.0×10⁻⁶/° C., cracks due to a volume difference occur, or a bonding failure occurs between the first lens p1 and the second lens p2.

For example, a material having a coefficient of thermal expansion of 7.2×10⁻⁶/° C. may be used as the first lens material al, and a material having a coefficient of thermal expansion of 7.1×10⁻⁶/° C. may be used as the second lens material a2.

In addition, it is preferable that the first lens material al is formed of a material having a Tg (i.e., glass transition temperature) point higher than a Tg point of the second lens material a2 by more than 50 degrees.

The reason is that when the Tg point of the first lens material al is lower than the Tg point of the second lens material a2, or is higher than the Tg point of the second lens material a2 by less than 50 degrees, a phenomenon of deformation or cracking of the first lens p1 occurs in step S300 of molding a second lens.

In addition, it is preferable that a difference between a refractive index Nd of the first lens material al and a refractive index Nd of the second lens material a2 exceeds 0.15, because this is the smallest possible difference in design for correction of chromatic aberration of lenses, and the greater the difference in the refractive index between the first lens material a1 and the second lens material a2, the more advantageous it is to design.

In addition, step S300 of molding a second lens may be repeated a number of times, and an achromatic lens in which three or more lenses are integrally combined with each other may be provided.

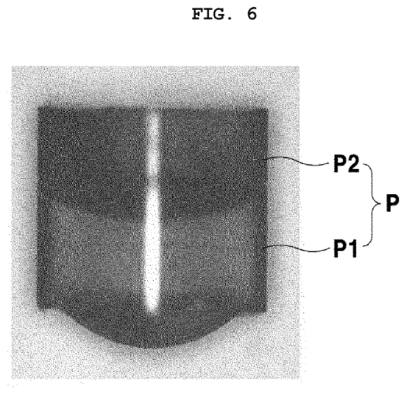

In addition, the present invention further provides an achromatic lens p manufactured by the method of manufacturing an achromatic lens according to the present invention, as shown in FIG. 6.

The achromatic lens p is provided with the first lens p1 and the second lens p2 formed integrally, and does not have a form that is physically bonded by a separate instrument or bonded by an adhesive.

In addition, the present invention may be provided as a medical endoscope provided with the achromatic lens p.

As described above, according to the method of manufacturing an achromatic lens of the present invention, since the first lens and the second lens are integrally molded by mold processing, compared with the conventional method of manufacturing an achromatic lens by using epoxy bonding, a phenomenon in which chromatic aberration occurs depending on physical properties, discharge amount, a bonding error of the epoxy resin, and the like may be eliminated, and compared with the manufacturing method of an achromatic lens by using the conventional instrument, the phenomena in which cracks, breaks, and the like occur due to the first lens and the second lens colliding with each other by the separate instrument, and chromatic aberration is generated due to the alignment error are prevented, whereby there is an advantage of having excellent chromatic aberration correction ability.

Furthermore, when the achromatic lens manufactured according to the present invention is applied as a medical endoscope, since an adhesive such as an epoxy resin is not used, there is an effect in that the concerns of harmfulness to the human body may be resolved.

As described above, the present invention has been illustrated and described with reference to preferred exemplary embodiments, but is not limited to the above-described exemplary embodiments, and various changes and modifications can be embodied by those skilled in the art to which the present invention belongs without departing from the spirit of the present invention.

What is claimed is:

1. A method of manufacturing an achromatic lens, the method comprising:
preparing molds comprising a sleeve mold provided with a cylindrical through-hole at a central part thereof, a lower mold fitted at an upper end thereof to a lower part of the through-hole of the sleeve mold, and a first upper mold and a second upper mold pressed downward from an upper part of the sleeve mold;
molding a first lens by placing a first lens material on an upper surface of the lower mold and pressing the first upper mold downward; and
molding a second lens by placing a second lens material on an upper surface of the first lens and pressing the second upper mold downward, thereby molding the achromatic lens in which the second lens is integrally molded on the first lens.

2. The method of claim 1, wherein the upper surface of the lower mold is provided with a planar shape, a convex part having an aspherical-surface shape, or a groove part having an aspherical-surface shape.

3. The method of claim 1, wherein the lower surface of the first upper mold is provided with a convex part having an aspherical-surface shape.

4. The method of claim 1, wherein the lower surface of the second upper mold is provided with a planar shape, a convex part having an aspherical-surface shape, or a groove part having an aspherical-surface shape.

5. The method of claim 1, wherein the first lens material and the second lens material have a difference in coefficient of thermal expansion of less than 1.0×10⁻⁶/° C.

6. The method of claim 1, wherein a material having a Tg point higher than a Tg (i.e., glass transition temperature) point of the second lens material by more than 50 degrees is used as the first lens material.

7. The method of claim 1, wherein a difference between a refractive index (Nd) of the first lens material and a refractive index (Nd) of the second lens material exceeds 0.15.

8. An achromatic lens comprising:
a first lens and a second lens provided integrally therein,
wherein the achromatic lens is manufactured by a method of manufacturing the achromatic lens of claim 1.

9. A medical endoscope comprising:
an achromatic lens of claim 8.
